(19) United States
(12) Patent Application Publication
(10) Pub. No.: US 2003/0060421 A1
(43) Pub. Date: Mar. 27, 2003

(54) Kits and compositions supporting intracranial perfusions

(75) Inventors: David P. Hesson, Malvern, PA (US); Glenn David Frazer, Wynnwood, PA (US); Timothy J. Pelura, Malvern, PA (US)

Correspondence Address: DECHERT, P.O. Box 5218, Princeton, NJ, 08543, US

(73) Assignee: Neuron Therapeutics, Inc.

(21) Appl. No.: 09/908,985

(22) Filed: Jul. 19, 2001

(51) Int. Cl. A61K 31/70; A61K 31/198; A61K 31/02

(52) U.S. Cl. 514/23; 514/561; 514/759

(57) ABSTRACT

Provided, among other things, is a kit providing pre-measured amounts of components to form a fluorocarbon nutrient emulsion capable of carrying oxygen to living tissue, the kit comprising: constituent solutions, emulsions or particle compositions, which are the constituent compositions, containing pre-measured amounts of components for making the fluorocarbon nutrient emulsion, the constituent compositions comprising: poly-fluorinated, oxygen-carrying compound; a physiologically acceptable emulsifying agent effective to emulsify the polymer; a nutrient-providing effective amount of carbohydrate; nutrient-providing effective amounts of amino acids or amino acid precursors; an oncotic agent in amount effective to provide, in conjunction with the other components of the solution, a physiologically acceptable oncotic pressure; and sufficient salts and buffering agents to provide a physiological osmotic pressure and physiologically appropriate concentrations of potassium and sodium ions; wherein constituent compositions are selected to allow for sufficient stability of the components to allow for commercial marketing of the kit.

This application claims the priority of U.S. Provisional Application No. 60/______ (pursuant to a petition converting U.S. patent application Ser. No. 09/619,414 to a provisional).

The present invention relates to compositions and kits for providing nutrients and oxygen to tissue, including cerebral tissue, and to associated methods.

Focal cerebral ischemia, or stroke, is the reduction or loss of blood flow to an area of cerebral tissue, denying the tissue sufficient oxygen and other metabolic resources. Similarly, during Traumatic Brain Injury (TBI) and Spinal Cord Injury (SCI) the tissues are also denied sufficient oxygen and other metabolic resources to carry out normal function or survive. Technology that has been explored by Osterholm has identified the cerebral spinal pathway, a connected system of cerebral ventricles and subarachnoid spaces of the brain and spinal cord, as an alternative pathway for delivering oxygen and nutrients to the tissue potentially affected by stroke. This stratagem has been shown in animal models for stroke to remarkably limit damage caused by focal cerebral ischemia.

The approach operates by placing a ventricular catheter into a lateral cerebral ventricle for use in administering an oxygenated fluorocarbon nutrient emulsion into the cerebral spinal pathway. The oxygenated fluorocarbon nutrient emulsion typically is made up of an emulsified fluorocarbon composition, where the fluorocarbon efficiently binds oxygen, and preferably carbon dioxide as well. The composition typically further contains additional nutrients. A second catheter is placed to allow drainage of fluid in the cerebral spinal pathway as needed in view of the injected fluorocarbon composition.

Applicants' recent experience indicates that nutrient compositions for use in the brain favorably contain no more than very limited amounts of all of glutamic acid, glutamine and glycine. During Traumatic Brain Injury (TBI) and Spinal Cord Injury (SCI) the tissues are also denied sufficient oxygen and other metabolic resources to carry out normal function or survive. Favorably, such compositions contain nutritionally useful amounts of arginine, histidine, leucine, lysine, methionine, phenylalanine, threonine and valine.

In seeking to package the components for such nutrient compositions, Applicants have noted that a number of combinations of components lack the stability needed for use as a commercial process. On the other hand, compounding from more basic components at or near the site and time of actual use increases the risk of error and microbial contamination. Applicants have identified combinations of constituent compositions that are adapted for use with automated compounding equipment that uses metered pumps to dispense the appropriate component amounts for making a fluorocarbon nutrient emulsion. These constituent compositions have sufficient stability to allow kits of the constituent compositions to be prepared under pharmaceutical Current Good Manufacturing Practices (as defined under title 21 of the Code of Federal Regulations, 2000 publication) and remain stable for months or years until final compounding.

Another recent discovery is the importance of having kits for preparing vehicle compositions that lack the fluorocarbon. The vehicle kit is preferably matched in most constituent compositions, helping to facilitate standardized preparation, but further includes constituent compositions designed to equalize salts that would be provided by the missing fluorocarbon-containing composition. The vehicle composition is favorably used during initial setup of the cerebral spinal perfusion, allowing a more convenient material to be used while seeking to establish a perfusion pathway.

### SUMMARY OF THE INVENTION

Provided is a kit providing pre-measured amounts of components to form a fluorocarbon nutrient emulsion capable of carrying oxygen to living tissue, the kit comprising: constituent solutions, emulsions or particle compositions, which are the constituent compositions, containing pre-measured amounts of components for making the fluorocarbon nutrient emulsion, the constituent compositions comprising: poly-fluorinated, oxygen-carrying compound; a physiologically acceptable emulsifying agent effective to emulsify the polymer; a nutrient-providing effective amount of carbohydrate; nutrient-providing effective amounts of amino acids or amino acid precursors; an oncotic agent in amount effective to provide, in conjunction with the other components of the solution, a physiologically acceptable oncotic pressure; and sufficient salts and buffering agents to provide a physiological osmotic pressure and physiologically appropriate concentrations of potassium and sodium ions; wherein constituent compositions are selected to allow for sufficient stability of the components to allow for commercial marketing of the kit.

Further provided is a kit providing pre-measured amounts of components to form a fluorocarbon nutrient emulsion capable of carrying oxygen to living tissue, the kit comprising: constituent solutions, emulsions or particle compositions, which are the constituent compositions, containing pre-measured amounts of components for making the fluorocarbon nutrient emulsion, the constituent compositions comprising: a first constituent composition comprising an emulsion of poly-fluorinated, oxygen-carrying compound; a second constituent composition comprising a solution of sodium and potassium salts; a third constituent composition comprising a solution of a nutrient-providing effective amount of glucose; a fourth constituent composition comprising a solution of an oncotic agent in amount effective to provide, in conjunction with the other components of the fluorocarbon nutrient emulsion, a physiologically acceptable oncotic pressure; a fifth constituent composition comprising solution of nutrient-providing effective amounts of amino acids; and a sixth constituent composition comprising a nutrient-providing effective amount of α-ketoglutaric acid.

Further provided is a kit providing pre-measured amounts of components to form a fluorocarbon nutrient emulsion capable of carrying oxygen to living tissue, the kit comprising: constituent solutions, emulsions or particle compositions, which are the constituent compositions, containing pre-measured amounts of components for making the fluorocarbon nutrient emulsion, the constituent compositions comprising: a first constituent composition comprising an emulsion of poly-fluorinated, oxygen-carrying compound (and optionally a nutrient-providing effective amount of α-ketoglutaric acid); a second constituent composition comprising a solution of sodium, potassium, magnesium and calcium salts (and optionally a nutrient-providing effective amount of glucose); a third constituent composition comprising a solution of oncotic agent in an amount effective to provide, in conjunction with the other components of the fluorocarbon nutrient emulsion, a physiologically acceptable oncotic pressure; and a fourth constituent composition comprising solution of a nutrient-providing effective amounts of amino acids. In a preferred embodiment, either the second constituent composition comprises a nutrient-providing effective amount of glucose or the kit comprises a fifth constituent composition comprising a nutrient-providing effective amount of glucose. In some embodiments, the second, fourth or fifteh constituent composition can be dried, preferably by lyophilization, and adapted to be diluted in a pre-determined amount of water before use.

Further provided is a vehicle kit providing pre-measured amounts of components to form a vehicle corresponding to a fluorocarbon nutrient emulsion formed from a corresponding fluorocarbon nutrient emulsion kit, the vehicle kit comprising the following separate vehicle kit compositions: all the first constituent compositions but the emulsified poly-fluorinated, oxygen-carrying compound composition; and supplement constituent compositions comprising one or more components effective to supply the sodium or potassium ions that would be provided by the emulsified poly-fluorinated, oxygen-carrying compound composition. The corresponding fluorocarbon nutrient emulsion kit comprising constituent solutions, emulsions or particle compositions, which are the first constituent compositions, containing pre-measured amounts of components for making the fluorocarbon nutrient emulsion, the first constituent compositions made up of: (a) poly-fluorinated, oxygen-carrying compound; (b) a phospholipid emulsifying agent effective to emulsify the poly-fluorinated, oxygen-carrying compound, wherein the poly-fluorinated, oxygen-carrying compound and the phospholipid emulsifying agent are supplied in one first constituent composition wherein the poly-fluorinated, oxygen-carrying compound is emulsified by the phospholipid emulsifying agent, this emulsified poly-fluorinated, oxygen-carrying compound composition providing a portion of sodium or potassium ions of the fluorocarbon nutrient emulsion; (c) a nutrient-providing effective amount of carbohydrate; (d) nutrient-providing effective amounts of amino acids or amino acid precursors; (e) an oncotic agent in amount effective to provide, in conjunction with the other components of the fluorocarbon nutrient emulsion, a physiologically acceptable oncotic pressure; and (f) sufficient salts and buffering agents to provide a physiological osmotic pressure and physiologically appropriate concentrations of potassium and sodium ions;

In one embodiment, the vehicle kit compositions of the corresponding fluorocarbon nutrient emulsion kit comprise: (1) a first constituent composition comprising an emulsion of poly-fluorinated, oxygen-carrying compound; (2) a first constituent composition comprising a solution of sodium, potassium, magnesium and calcium salts; (3) a first constituent composition comprising a solution of a nutrient-providing effective amount of glucose; (4) a first constituent composition comprising a solution of an oncotic agent in amount effective to provide, in conjunction with the other components of the fluorocarbon nutrient emulsion, a physiologically acceptable oncotic pressure; (5) a first constituent composition comprising a solution of nutrient-providing effective amounts of amino acids; and (6) a first constituent composition comprising a nutrient-providing effective amount of α-ketoglutaric acid, whereby the vehicle kit compositions comprise first constituent compositions (b) through (f) and at least one supplement constituent composition.

In another embodiment, the vehicle kit compositions of the corresponding fluorocarbon nutrient emulsion kit comprise: (1) a first constituent composition comprising an emulsion of poly-fluorinated, oxygen-carrying compound (and optionally a nutrient-providing effective amount of a-ketoglutaric acid); (2) a first constituent composition comprising a solution of sodium, potassium, magnesium and calcium salts (and optionally a nutrient-providing effective amount of glucose); (3) a first constituent composition comprising a solution of the oncotic agent in amount effective to provide, in conjunction with the other components of the fluorocarbon nutrient emulsion, a physiologically acceptable oncotic pressure; and (4) a first constituent composition comprising a solution of a nutrient-providing effective amounts of amino acids, whereby the vehicle kit compositions comprise first constituent compositions (2) through (4) and at least one supplement constituent composition. Preferably, the vehicle kit comprises a first vehicle kit composition comprising a solution of (i) sodium, potassium, magnesium and calcium salts, (ii) a nutrient-providing effective amount of α-ketoglutaric acid, and (iii) a nutrient-providing effective amounts of amino acids; a second vehicle kit composition comprising a solution of the oncotic agent in amount effective to provide, in conjunction with the other components of the fluorocarbon nutrient emulsion, a physiologically acceptable oncotic pressure; and a third vehicle kit composition comprising a solution of a nutrient-providing effective amount of glucose.

Further provided is a method of delivering a fluorocarbon nutrient emulsion to neural tissue of an animal having a cerebrospinal pathway, the method comprising: (a) inserting a first catheter at a first point directed to deliver fluid to a cerebral ventricle; (b) inserting a second catheter at a second point lower in the cerebrospinal pathway, which point is adapted to drain excess fluid due to fluid insertion through the first catheter; (c) inserting through the first catheter a vehicle solution adapted to be physiologically compatible with the fluorocarbon nutrient emulsion, wherein the vehicle solution lacks sufficient oxygen carrying capacity to be capable of carrying a respiration-supporting amount of oxygen; (d) confirming with the vehicle solution the existence of a perfusion pathway from the first catheter to the second catheter; (e) if necessary, repositioning one or both of the catheters and repeating step (d) until a perfusion pathway is confirmed; and (f) once a perfusion pathway is confirmed, inserting an oxygenated fluorocarbon nutrient emulsion through the first catheter.

Still further provided is a fluorocarbon nutrient emulsion capable of carrying a oxygen to living tissue or a kit of pre-measured components for such a solution, the solution or kit comprising: a poly-fluorinated, oxygen-carrying compound; a physiologically acceptable emulsifying agent effective to emulsify the poly-fluorinated, oxygen-carrying compound; and nutrient-providing effective amounts of amino acids or amino acid precursors, wherein the solution or kit is essentially lacking in glutamic acid, glutamine and glycine.

Also provided is a nutrient solution or a kit of pre-measured components for such a solution, the solution or kit comprising: a nutrient-providing effective amount of carbohydrate; an oncotic agent in amount effective to provide, in conjunction with the other components of the solution, a physiologically acceptable oncotic pressure; and nutrient-providing effective amounts of amino acids or amino acid precursors including arginine, histidine, leucine, isoleucine, lysine, methionine, phenylalanine, threonine and valine, wherein the solution or kit is essentially lacking in glutamic acid, glutamine and glycine.

Still further provided is a fluorocarbon nutrient emulsion capable of carrying a oxygen to living tissue or a kit of pre-measured components for such a solution, the solution or kit comprising: a poly-fluorinated, oxygen-carrying compound; a physiologically acceptable emulsifying agent effective to emulsify the poly-fluorinated, oxygen-carrying compound; and nutrient-providing effective amounts of amino acids or amino acid precursors, including at least one citric acid, cis-aconitic acid, isocitric acid, succinic acid, fumaric acid, malic acid or oxaloacetic acid or a pharmaceutically acceptable salt thereof, wherein the solution or kit is essentially lacking in glutamic acid and glutamine.

Also provided is a nutrient solution or a kit of pre-measured components for such a solution, the solution or kit comprising: a nutrient-providing effective amount of carbohydrate; an oncotic agent in amount effective to provide, in conjunction with the other components of the solution, a physiologically acceptable oncotic pressure; and nutrient-providing effective amounts of amino acids or amino acid precursors including arginine, histidine, leucine, isoleucine, lysine, methionine, phenylalanine, threonine, valine, and at least one of citric acid, cis-aconitic acid, isocitric acid, succinic acid, fumaric acid, malic acid or oxaloacetic acid or a pharmaceutically acceptable salt thereof, wherein the solution or kit is essentially lacking in glutamic acid and glutamine.

Further provided is a method of irrigating exposed cerebral-spinal tissue comprising irrigating with a solution comprising: a nutrient-providing effective amount of carbohydrate; an oncotic agent in amount effective to provide, in conjunction with the other components of the solution, a physiologically acceptable oncotic pressure; and nutrient-providing effective amounts of amino acids or amino acid precursors including arginine, histidine, leucine, isoleucine, lysine, methionine, phenylalanine, threonine, valine, and at least one of citric acid, cis-aconitic acid, isocitric acid, α-ketoglutaric acid succinic acid, fumaric acid, malic acid or oxaloacetic acid or a pharmaceutically acceptable salt thereof, wherein the solution or kit is essentially lacking in glutamic acid and glutamine. The surgery can comprise: opening an animal to provide access to cerebral spinal tissue; irrigating the accessed cerebral spinal tissue; and conducting the surgery. Or it can comprise: irrigating the exposed cerebral spinal tissue of an animal; conducting the surgery; and closing the animal to end the exposed state of the spinal tissue.

Still further provided is a method of delivering a fluorocarbon nutrient emulsion to neural tissue of an animal having a cerebrospinal pathway, the method comprising: oxygenating an emulsion of a poly-fluorinated, oxygen-carrying compound at a temperature from within ±1° C. of the physiological intracranial temperature (T₀) of the animal; reducing the temperature of the oxygenated emulsion by at least 2° C.; and perfusing the reduced temperature emulsion through at least a portion of the cerebral spinal pathway. Also provided is a method of delivering a physiologically acceptable solution or suspension (such as an emulsion) to neural tissue of an animal having a cerebrospinal pathway, the method comprising: conditioning the solution or suspension at temperature from within ±1° C. of the physiological intracranial temperature (T₀) of the animal; reducing the temperature of the conditioned solution or suspension by at least 2° C.; and perfusing the reduced temperature solution or suspension through at least a portion of the cerebral spinal pathway.

The devices and methods of the invention can be used to treat stroke, TBI, SCI or any other condition likely to deprive cerebral spinal tissue of needed oxygen or nutrients.

### BRIEF DESCRIPTION OF THE DRAWINGS

FIG. 1 displays the catheter placements for a venticulo-lumbar perfusion.

FIG. 2 shows an exemplary perfusion device.

### DETAILED DESCRIPTION OF THE INVENTION

Fluorocarbon Nutrient Emulsions and Kits

The suspensions of poly-fluorinated, oxygen-carrying compound used to deliver oxygen, and preferably remove carbon dioxide from cerebral-spinal tissue pursuant to various methods described herein have been found to be relatively unstable for storage. Even the non-emulsion components cannot be stored as a complete mixture for sufficient periods of time without precipitates and other undesirable components forming. Readily standardized, stable solutions or suspensions have now been discovered. These solutions or suspensions can be delivered to automated compounding equipment, such as an Automix compounding device from Clintec Nutrition Company, Deerfield, Ill. In preferred embodiments, the fluorocarbon nutrient emulsions contain a protein-based oncotic agent, which is stored separately from the carbohydrate component. The oncotic agent is preferably also stored separate from any keto-containing component. Similarly, the amino acid components are preferably stored separate from the carbohydrate component, and preferably separate from any keto-containing component.

When present, the constituents of the fluorocarbon nutrient emulsion of the invention are preferably in amounts as described in the table below:

| | | | Still More |
|---|---|---|---|
| | | More | Preferred |
| | Preferred | Preferred | Range |
| Component | Range | Range | or Amount |
| Poly-Fluorinated, | 5-15 | 9-11 | 9.5-10-5 |
| Oxygen-Carrying Compound, | | | |
| % v/v | | | |
| Phospholipid, mg/mL | 8-14 | 10-13 | 11.5 |
| Albumin, g/dL, | 0.05-2.0 | 1.5-1.9 | 1.67 |
| α-Ketoglutaric Acid, μg/mL | 5-40 | 22-28 | 25 |
| Amino Acids, μg/mL | | | |
| L-Isoleucine + L-Leucine | 5-50 | 11-23 | 17.5 |
| L-Valine | 5-50 | 11-22 | 16.6 |
| L-Alanine | 5-50 | 19-38 | 28.6 |
| L-Serine | 5-50 | 16-33 | 24.6 |
| L-Histidine | 2-20 | 7-14 | 10.3 |
| L-Methionine | 0.1-5 | 1.4-2.8 | 2.1 |
| L-Phenylalanine + L-Lysine | 5-50 | 23-47 | 35.3 |
| L-Threonine + L-Arginine | 5-50 | 32-64 | 48.3 |
| L-Tyrosine | 1-20 | 5-11 | 7.9 |
| Na⁺, mM | 135-150 | 137-147 | 147 |
| K⁺, mM | 2.5-4.0 | 2.7-3.9 | 2.9 |
| Cl⁻, mM | 110-135 | 116-135 | 130 |
| Ca⁺², mM | 1.0-1.6 | 1.0-1.5 | 1.15 |
| Mg⁺², mM | 0.8-1.6 | 1.0-1.5 | 1.12 |
| Glucose (dextrose), mg/dL | 10-150 | 30-100 | 94 |

The pH of the emulsion, or vehicle, is in the physiological range, such as about 7.3. In one embodiment, the amino acids include tryptophan.

One exemplary kit for making fluorocarbon nutrient emulsion containing eight constituent compositions is as set forth in the table below for a 1200 mL unit of the emulsion. Though the constituent compositions are provided to the mixing process in somewhat greater volume than listed in the table below (e.g., 515 mL for 500 mL), the amounts listed below are normalized to the 1200 mL of the final unit.

| | | | Amount |
|---|---|---|---|
| | | Constituent Compositions | g/unit |
| 1. | F44E | t-Bis-perfluorobutyl ethylene | 200 |
| | Emulsion | NaCl, USP | 2.7 |
| | 500 mL* | NaHCO₃, USP | 0.85 |
| | | Purified egg yolk phospholipid, | 13.8 |
| | | K⁺ 2.7 mM | |
| 2. | Salt Annex | NaCl, USP | 4.09 |
| | 100 mL | KCl, USP | 0.15 |
| | | MgCl₂—6H₂O, USP | 0.24 |
| | | CaCl₂—2H₂O, USP | 0.18 |
| 3. | 20 mL | Dextrose, USP | 1 |
| 4. | 100 mL | Albumin (Human), USP (20%) | 20 |
| | | NaCl 145 mM/L | |
| 5. | Amino | L-lysine HCl, USP | 0.0032 |
| | Acid | L-alanine, USP | 0.0034 |
| | Annex | L-serine, USP | 0.0030 |
| | 100 mL | L-threonine, USP | 0.0036 |
| | | L-arginine, USP | 0.0022 |
| | | L-leucine, USP | 0.0015 |
| | | L-isoleucine, USP | 0.0006 |
| | | L-valine, USP | 0.0020 |
| | | L-phenylalanine, USP | 0.0010 |
| | | L-tyrosine, USP | 0.0010 |
| | | L-histidine, USP | 0.0012 |
| | | L-methionine, USP | 0.0003 |
| | | KCl, USP | 0.3 |
| | | NaH₂PO₄, USP | 4.1 |
| | | Na₂HPO₄, USP | 0.61 |
| 6. | 1 mL | α-ketoglutaric acid | 0.030 |
| 7. | 16 mL | NaHCO₃, USP [1 M] | 1.344 |
| 8. | QS | Sterile Water for Injection, USP | |

To make a vehicle kit, supplement constituent compositions containing the sodium and potassium are provided to deliver the sodium and potassium ions otherwise provided by constituent composition #1. In the above example kit, the α-ketoglutaric acid component can be stored as a dry powder, which is dissolved in, for example, sterile water before use.

A second exemplary kit for making fluorocarbon nutrient emulsion containing four constituent compositions is as set forth in the table below normalized for a 3,000 mL unit of the emulsion.

| | | | Amount |
|---|---|---|---|
| | | Constituent Compositions | g/unit |
| 1. | Stem | t-Bis-perfluorobutyl ethylene | 500 |
| | Emulsion | NaHCO₃, USP | 5.48 |
| | 1000 mL | NaH₂PO₄, USP | 0.11 |
| | | α-ketoglutaric acid | 0.0750 |
| | | Purified egg yolk phospholipid, | 34.50 |
| | | K⁺ 2.7 Mm | |
| 2. | Salt Annex | NaCl, USP | 16.97 |
| | 1000 mL | KCl, USP | 0.39 |
| | | MgCl₂—6H₂O, USP | 0.61 |
| | | CaCl₂—2H₂O, USP | 0.45 |
| | | Dextrose, USP | 2.50 |
| 3. | 250 mL | Albumin (Human), USP (20%) | 100 |
| | | NaCl 145 mM/L | |
| 4. | Amino | L-lysine HCl, USP | 0.0083 |
| | Acid | L-alanine, USP | 0.0086 |
| | Annex | L-serine, USP | 0.0073 |
| | 75 mL | L-threonine, USP | 0.0089 |
| | | L-arginine, USP | 0.0056 |
| | | L-leucine, USP | 0.0039 |
| | | L-isoleucine, USP | 0.0014 |
| | | L-valine, USP | 0.0050 |
| | | L-phenylalanine, USP | 0.0026 |
| | | L-tyrosine, USP | 0.0024 |
| | | L-histidine, USP | 0.0029 |
| | | L-methionine, USP | 0.0008 |

A third exemplary kit for making fluorocarbon nutrient emulsion containing five constituent compositions is as set forth in the table below for a 1200 mL unit of the emulsion. The lyophilized compositions described below are reconstituted with water, preferably USP sterile water for injection, prior to addition into composition 1. The dilution amounts are 20 mL each for compositions 2 and 3, and 10 mL for composition 5

| | | | Amount |
|---|---|---|---|
| | | Constituent Compositions | g/unit |
| 1. | F44E | t-Bis-perfluorobutyl ethylene | 200 |
| | Emulsion | NaCl, USP | 2.7 |
| | 1050 mL* | NaHCO₃, USP | 2.19 |
| | | Purified egg yolk phospholipid, | 13.8 |
| | | K⁺ 2.7 mM | |
| 2. | Salt Annex | NaCl, USP | 4.09 |
| | Lyophilized | KCl, USP | 0.15 |
| | Powder, | MgCl₂—6H₂O, USP | 0.24 |
| | (4.66 g total) | CaCl₂—2H₂O, USP | 0.18 |
| 3. | Lyophilized | Dextrose, USP | 1 |
| | Powder | | |
| 4. | 100 mL | Albumin (Human), USP (20%) | 20 |
| | | NaCl 145 mM/L | |
| 5. | Amino | L-lysine HCl, USP | 0.0083 |
| | Acid-α- | L-alanine, USP | 0.0086 |
| | Ketoglutaric | L-serine, USP | 0.0073 |
| | Acid | L-threonine, USP | 0.0089 |
| | Annex, | L-arginine, USP | 0.0056 |
| | Lyophilized | L-leucine, USP | 0.0039 |
| | Powder | L-isoleucine, USP | 0.0014 |
| | | L-valine, USP | 0.0050 |
| | | L-phenylalanine, USP | 0.0026 |
| | | L-tyrosine, USP | 0.0024 |
| | | L-histidine, USP | 0.0029 |
| | | L-methionine, USP | 0.0008 |
| | | KCl, USP | 0.3 |
| | | NaH₂PO₄, USP | 4.1 |
| | | Na₂HPO₄, USP | 0.61 |
| | | α-ketoglutaric acid | 0.030 |

A forth exemplary kit for making fluorocarbon nutrient emulsion containing four constituent compositions is as set forth in the table below for a 1200 mL unit of the emulsion. The lyophilized compositions described below are reconstituted with water, preferably USP sterile water for injection, prior to addition into composition 1. The dilution amounts are 20 mL for composition 2 and 10 mL for composition 4.

| | | | Amount |
|---|---|---|---|
| | | Constituent Compositions | g/unit |
| 1. | F44E | t-Bis-perfluorobutyl ethylene | 200 |
| | Emulsion | NaCl, USP | 2.7 |
| | 1070 mL* | NaHCO₃, USP | 2.19 |
| | | Purified egg yolk phospholipid, | 13.8 |
| | | K⁺ 2.7 mM | |
| 2. | Salt Annex | NaCl, USP | 4.09 |
| | Lyophilized | KCl, USP | 0.15 |
| | Powder, | MgCl₂—6H₂O, USP | 0.24 |
| | (5.66 g total) | CaCl₂—2H₂O, USP | 0.18 |
| | | Dextrose, USP | 1 |
| 3. | 100 mL | Albumin (Human), USP (20%) | 20 |
| | | NaCl 145 mM/L | |
| 4. | Amino | L-lysine HCl, USP | 0.0083 |
| | Acid-α- | L-alanine, USP | 0.0086 |
| | Ketoglutaric | L-serine, USP | 0.0073 |
| | Acid | L-threonine, USP | 0.0089 |
| | Annex, | L-arginine, USP | 0.0056 |
| | Lyophilized | L-leucine, USP | 0.0039 |
| | Powder | L-isoleucine, USP | 0.0014 |
| | | L-valine, USP | 0.0050 |
| | | L-phenylalanine, USP | 0.0026 |
| | | L-tyrosine, USP | 0.0024 |
| | | L-histidine, USP | 0.0029 |
| | | L-methionine, USP | 0.0008 |
| | | KCl, USP | 0.3 |
| | | NaH₂PO₄, USP | 4.1 |
| | | Na₂HPO₄, USP | 0.61 |
| | | α-ketoglutaric acid | 0.030 |

A fifth exemplary kit for making fluorocarbon nutrient emulsion containing four constituent compositions is as set forth in the table below for a 1200 mL unit of the emulsion. The lyophilized composition described below is reconstituted with water, preferably USP sterile water for injection, prior to addition into composition 1. The dilution amount is 10 mL.

| | | | Amount |
|---|---|---|---|
| | | Constituent Compositions | g/unit |
| 1. | F44E | t-Bis-perfluorobutyl ethylene | 200 |
| | Emulsion | NaCl, USP | 2.7 |
| | 1030 mL* | NaHCO₃, USP | 2.19 |
| | | Purified egg yolk phospholipid, | 13.8 |
| | | K⁺ 2.7 mM | |
| 2. | Salt and | NaCl, USP | 4.09 |
| | Dextrose | KCl, USP | 0.15 |
| | Annex | MgCl₂—6H₂O, USP | 0.24 |
| | 50 mL | CaCl₂—2H₂O, USP | 0.18 |
| | | Dextrose, USP | 1 |
| 3. | 100 mL | Albumin (Human), USP (20%) | 20 |
| | | NaCl 145 mM/L | |
| 4. | Amino | L-lysine HCl, USP | 0.0083 |
| | Acid-α- | L-alanine, USP | 0.0086 |
| | Ketoglutaric | L-serine, USP | 0.0073 |
| | Acid | L-threonine, USP | 0.0089 |
| | Annex, | L-arginine, USP | 0.0056 |
| | Lyophilized | L-leucine, USP | 0.0039 |
| | Powder | L-isoleucine, USP | 0.0014 |
| | | L-valine, USP | 0.0050 |
| | | L-phenylalanine, USP | 0.0026 |
| | | L-tyrosine, USP | 0.0024 |
| | | L-histidine, USP | 0.0029 |
| | | L-methionine, USP | 0.0008 |
| | | KCl, USP | 0.3 |
| | | NaH₂PO₄, USP | 4.1 |
| | | Na₂HPO₄, USP | 0.61 |
| | | α-ketoglutaric acid | 0.030 |

To make a vehicle kit, supplement constituent compositions containing the sodium and potassium are provided to deliver the sodium and potassium ions otherwise provided by constituent composition #1. In a preferred embodiment, the supplement constituent components also provide the α-ketoglutaric acid provided by constituent composition #1.

To make a fluorocarbon nutrient emulsion kit with three constituent compositions, for example, one can provide the amino acid annex with the emulsified fluorocarbon composition.

In one embodiment, most of the constituent compositions are packaged in separate chambers of a multi-chambered bag, where the seams between the chambers can be broken by applying pressure to the chambers, thereby mixing the contents of the chambers. Such multi-chambered bags are available, for example, as Compleven™ multi-chambered bags, which are marketed by Fresenius Kabi, Uppsala, Sweden. Such bags are preferably manufactured to provide reduced oxygen and carbon dioxide permeability. Such bags can be constructed of, for example, ethylene vinyl acetate (EVA) or polyvinylchloride (PVC). An outer bag, or outer layer of polymer can be provided to reduce oxygen permeability. In many embodiments, where albumin is used as the oncotic agent, the chamber used to house the oncotic agent is lined with a non-plasticized polymer such as polyester films, (including the polyester films and suitable multi-layered films based on a polyester support marketed by E.I. Dupont du Nemours and Company, Wilmington, Del. as Mylar® film), polyester-based multi-layer films having a metal foil layer (again marketed as a form of Mylar® film) polyolefin or a metal foil, such as an aluminum foil (itself not coated with a plasticized polymer). Alternatively, the oncotic agent can be stored separately, for example in glass, and injected into the chambers at the time of use. The multi-chambered bag is thus favorably provided with an injection port.

As mentioned, with the multi-chambered bag, pressure can be used to break the barriers between chambers to allow the contents to mix, with the contents mixing to provide the appropriate concentrations. The appropriate concentration is that of the final fluorocarbon nutrient emulsion adjusted for the dilutions or additions provided by any separate fourth constituent composition.

Favorably, when not using the multi-chambered bag, the kit is provided in standardized packaging, with designated slots for each constituent composition. Favorably, each constituent composition can have highly visible unique markings, which markings can be keyed to markings provided for at the automated compounding equipment to assure that each constituent composition is applied to that equipment consistent with the instrument's programming for compounding the fluorocarbon nutrient emulsion. Such markings can include color codes, color combinations, bar codes, and the like.

When packaged in a multi-chambered bag, the kits of the invention are preferably packaged in a gas-impermeable such bag. Since flexible plastics are not, in an absolute sense, impermeable to all gas transmission, “gas-impermeable” refers to a sufficiently reduced transmission so as to allow storage for months or more without substantial deterioration in quality due to oxygen or carbon dioxide intrusion or water vapor loss. For example, preferably the gas permeability of the bag, or the bag in combination with an outer bag enveloping the mult-chambered bag has an oxygen permeability (measured under ASTM D3985) or carbon dioxide permeability of 10 cc/m²·day·atm or less, preferably 1.0, 0.5 or 0.2 cc/m²·day·atm or less. For example, the multi-compartment bags can be enclosed in Mylar® MC2 film (DuPont), which is a polyester film with a vacuum deposited layer of aluminum on one side and overcoated on both sides with a heat sealable polyvinyl dichloride copolymer. The film has excellent oxygen, moisture and light barrier properties (oxygen permeability of 0.15 cc/m²·day·atm), and is available in 50 gauge (34,900 in²/lb) and 100 gauge (19,700 in²/lb). Strong materials with low oxygen permeability can also be made with Kevlar® (DuPont). Kevlar® polymer materials consist of long molecular chains produced from poly-paraphenylene terephthalamide. The chains are highly oriented with strong interchain bonding.

The emulsion (or the corresponding vehicle) preferably comprises nutrient-providing effective amounts of arginine, histidine, leucine, lysine, methionine, phenylalanine, threonine and valine. Preferably the emulsion (or the corresponding vehicle) is essentially lacking in glutamic acid, glutamine and glycine. Preferably, the salts provided include physiologically suitable amounts of potassium and sodium salts, as well, preferably, as calcium or magnesium salts. Preferably, glutamic acid and glutamine are avoided, but a precursor is provided in the form of at least one of citric acid, cis-aconitic acid, isocitric acid, α-ketoglutaric acid, succinic acid, fumaric acid, malic acid or oxaloacetic acid, or a pharmaceutically acceptable salt thereof. In some embodiments, the precursor is provided in the form of at least one of citric acid, cis-aconitic acid, isocitric acid, succinic acid, fumaric acid, malic acid or oxaloacetic acid, or a pharmaceutically acceptable salt thereof. In other embodiments, the precursor is provided in the form of α-ketoglutaric acid or a pharmaceutically acceptable salt thereof.

It should be recognized that any component molecule, such as an amino acid, used in the invention which has a salt form, can be utilized in such a salt form so long as the counterion does not disrupt the physiological or pharmacologic acceptability of a nutrient emulsion or vehicle solution.

Vehicle Solutions and Kits

As alluded to above, the fluorocarbon nutrient emulsions can be matched to vehicle solutions, which preferably are matched in all physiologically significant ions, nutrients and oncotic agents. Where kits for vehicle are provided, these preferably share most of the constituent compositions of kits for the corresponding emulsion, with one or more new constituent compositions provided to supply significant components absent due to the absence of the emulsion of poly-fluorinated oxygen-carrying compound.

One exemplary kit for making fluorocarbon nutrient emulsion vehicle containing nine constituent compositions is as set forth in the table below for a 1200 mL unit of the emulsion. This kit is substantially matched to the first exemplary fluorocarbon nutrient emulsion kit.

| | | | Amount |
|---|---|---|---|
| | | Constituent Compositions | g/unit |
| 1. | Salt | NaCl, USP | 4.09 |
| | Annex | KCl, USP | 0.15 |
| | 100 mL | MgCl₂—6H₂O, USP | 0.24 |
| | | CaCl₂—2H₂O, USP | 0.18 |
| 2. | 20 mL | Dextrose | 1 |
| 3. | 100 mL | Albumin (Human), USP (20%) | 20 |
| | | NaCl 145 mM/L | |
| 4. | Amino | L-lysine HCl, USP | 0.0032 |
| | Acid | L-alanine, USP | 0.0034 |
| | Annex | L-serine, USP | 0.0030 |
| | 10 mL | L-threonine, USP | 0.0036 |
| | | L-arginine, USP | 0.0022 |
| | | L-leucine, USP | 0.0015 |
| | | L-isoleucine, USP | 0.0006 |
| | | L-valine, USP | 0.0020 |
| | | L-phenylalanine, USP | 0.0010 |
| | | L-tyrosine, USP | 0.0010 |
| | | L-histidine, USP | 0.0012 |
| | | L-methionine, USP | 0.0003 |
| | | KCl, USP | 0.3 |
| | | NaH₂PO₄, USP | 4.1 |
| | | Na₂HPO₄, USP | 0.61 |
| 5. | 1 mL | α-ketoglutaric acid | 0030 |
| 6. | 25 mL | NaHCO₃ | 2.19 |
| 7. | 423 mL | Sterile Water for Injection, USP | |
| 8. | 300 mL | Sterile Saline, 0.9% w/v, USP | 3.21 (NaCl) |
| 9. | 0.465 | KCl, 2 mEq/mL, USP | 0.075 |

Constituent compositions nos. 8 and 9 provide the sodium and potassium ions that otherwise would be provided by the fluorocarbon nutrient emulsion.

A second exemplary kit for making fluorocarbon nutrient emulsion vehicle containing nine constituent compositions is as set forth in the table below for a 3,000 mL unit of the emulsion. This kit is substantially matched to the second exemplary fluorocarbon nutrient emulsion kit.

| | | | Amount |
|---|---|---|---|
| | | Constituent Compositions | g/unit |
| 1. | Vehicle | NaHCO₃, USP | 5.48 |
| | Annex | NaH₂PO₄, USP | 0.19 |
| | 1000 mL | α-ketoglutaric acid | 0.11 |
| | | K⁺ from Lecithin (KCl) 2.7 mM | 0.0750 |
| | | Lecithin, purified egg yolk | |
| 2. | Salt | NaCl, USP | 16.97 |
| | Annex | KCl, USP | 0.39 |
| | 1000 mL | MgCl₂—6H₂O₂, USP | 0.61 |
| | | CaCl₂—2H₂O, USP | 0.45 |
| | | Dextrose, USP | 2.50 |
| 3. | 250 mL | Albumin (Human), USP (20%) | 100 |
| | | NaCl 145 mM/L | |
| 4. | Amino | L-lysine HCl, USP | 0.0083 |
| | Acid | L-alanine, USP | 0.0086 |
| | Annex | L-serine, USP | 0.0073 |
| | 750 mL | L-threonine, USP | 0.0089 |
| | | L-arginine, USP | 0.0056 |
| | | L-leucine, USP | 0.0039 |
| | | L-isoleucine, USP | 0.0014 |
| | | L-valine, USP | 0.0050 |
| | | L-phenylalanine, USP | 0.0026 |
| | | L-tyrosine, USP | 0.0024 |
| | | L-histidine, USP | 0.0029 |
| | | L-methionine, USP | 0.0008 |

A third exemplary kit for making fluorocarbon nutrient emulsion vehicle containing three constituent compositions is as set forth in the table below for a 1069 mL unit of the solution. Composition 3 is reconstituted with water, preferably USP sterile water for injection, prior to addition into composition 1. The dilution amount is 20 mL.

| | | | Amount |
|---|---|---|---|
| | | Constituent Compositions | g/unit |
| 1. | Vehicle | NaCl, USP | 7.30 |
| | Annex | KCl, USP | 0.225 |
| | Solution | MgCl₂—6H₂O, USP | 0.24 |
| | 949 mL | CaCl₂—2H₂O, USP | 0.18 |
| | | NaHCO₂, USP | 2.19 |
| | | L-lysine HCl, USP | 0.0083 |
| | | L-alanine, USP | 0.0086 |
| | | L-serine, USP | 0.0073 |
| | | L-threonine, USP | 0.0089 |
| | | L-arginine, USP | 0.0056 |
| | | L-leucine, USP | 0.0039 |
| | | L-isoleucine, USP | 0.0014 |
| | | L-valine, USP | 0.0050 |
| | | L-phenylalanine, USP | 0.0026 |
| | | L-tyrosine, USP | 0.0024 |
| | | L-histidine, USP | 0.0029 |
| | | L-methionine, USP | 0.0008 |
| | | KCl, USP | 0.3 |
| | | NaH₂PO₄, USP | 4.1 |
| | | Na₂HPO₄, USP | 0.61 |
| | | α-ketoglutaric acid | 0.030 |
| 2. | 100 mL | Albumin (Human), USP (20%) | 20 |
| | | NaCl 145 mM/L | |
| 3. | Dextrose | Dextrose, USP | 1 |
| | Lyophilized | | |
| | powder | | |

The invention provides a kit which is a jointly packaged combination of a fluorocarbon nutrient emulsion and a corresponding vehicle kit. Preferably the packaging places the components of the two kits in recognizably distinct locations, and used labeling that makes clear the constituent compositions of each sub-kit.

Poly-Fluorinated, Oxygen-Carrying Compounds

Poly-fluorinated, oxygen-carrying compounds are known in the art. The basic requirements are effectiveness in carrying a physiologically useful amount of oxygen. Factors involved in selecting preferred such compounds include oxygen capacity, tissue retention (preferably minimized), emulsion stability, toxicity, and the like. Such compounds are described, for example, in: Riess et al., “Design Synthesis and Evaluation of Fluorocarbons and Surfactants for In vivo Applications New Perfluoroalkylated Polyhydroxylated Surfactants”, Biomat. Artif Cells Artif Organs, 16:421-430 (1988); Riess, Reassessment of criteria for the Selection of Perfluorochemicals for Second-Generation Blood Substitutes: Analysis of Structure/Property Relationships, Artificial Organs 8:44-56 (1984); Riess, et al., Design, Synthesis and Evaluation of Fluorocarbons and Surfactants for In Vivo Applications New Perfluoralkylated Polyhydroxylated Surfactants, Biomat. Artif. Cells Artif Organs 16:421-430 (1988); Riess, et al., Solubility and Transport Phenomena in Perfluorochemicals Relevant to Blood Substitution and Other Biomedical Applications, Pure & Applied Chem., 54:2383-2406 (1982); Yamanouchi, et al., Quantitative Structure-In Vivo Half-Life Relationships of Perfluorochemicals for Use as Oxygen Transporters, Chem., Pharm. Bull., 33:1221-1231 (1985); Lowe, et al., Perfluorochemicals: Blood Substitutes and Beyond Adv. Mater, 3:87-93 (February, 1991); Riess, et al., Fluorocarbon-Based In Vivo Oxygen Transport and Delivery Systems Vox Sang, 61:225-239 (December 1991); and Weers, et al., U.S. Pat. No. 5,914,352.

Among preferred poly-fluorinated, oxygen-carrying compounds are those of the formula

CₘFₘ₊₁—CH═CH—CₙFₙ₊₁,

where m+n equals 6 to 10. Preferably, the double bond is trans. One preferred poly-fluorinated, oxygen-carrying compound is trans-Bis-perfluorobutyl ethylene (m and n each equal 4). Also preferred are those of the formula

CₘFₘ₊₁—O—CₙFₙ₊₁,

where m+n equals 6 to 9 (or 8). One of the perfluoro alkyls can be substituted with a halo from Br (preferably), Cl or I. Further preferred are those of the formula

CₘFₘ₊₁—R,

where m is 8 (or 10) to 12 and R is Br, Cl, I, or C₁-C₃ alkyl.

Method of Delivering Fluorocarbon Nutrient Emulsion

Despite the safety of the emulsions of poly-fluorinated, oxygen-carrying compound preferred for use in the invention, it has now been recognized as preferable to establish a flow pathway from the entry catheter (e.g., a ventricular catheter into a lateral ventricle of the brain) to an exit point at a different location in the cerebral spinal pathway (e.g., into the intrathecal space of the lumbar (L4-L5) region of the spine) without prematurely inserting the emulsion.

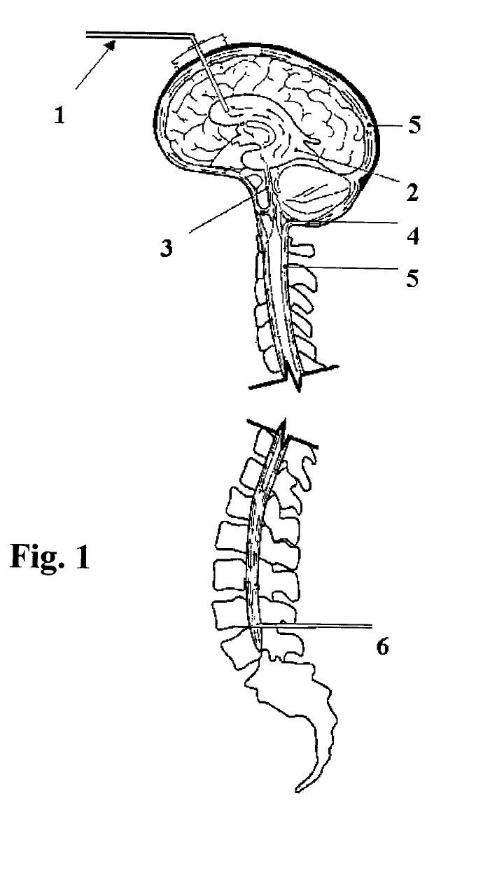

As illustrated in FIG. 1, a ventricular catheter 1 is inserted into a lateral ventrical 2. Via aqueduct 3, cisterna magna 4 and subarachnoid spaces 5, a flow pathway can be established to a lumbar outflow catheter 6. When the inflow and outflow catheters are established (typically with suitable controls to monitor intracranial and intraspinal pressure), vehicle can be used to establish the existence of a flow pathway (such as that illustrated) from the inflow catheter to the outflow catheter. Preferably, the vehicle is infused under gravity feed, with the pressure head designed to avoid excessive intracranial pressure. Once established, the vehicle can be substituted with the emulsion of poly-fluorinated, oxygen-carrying compound.

Exemplary Perfusion Device

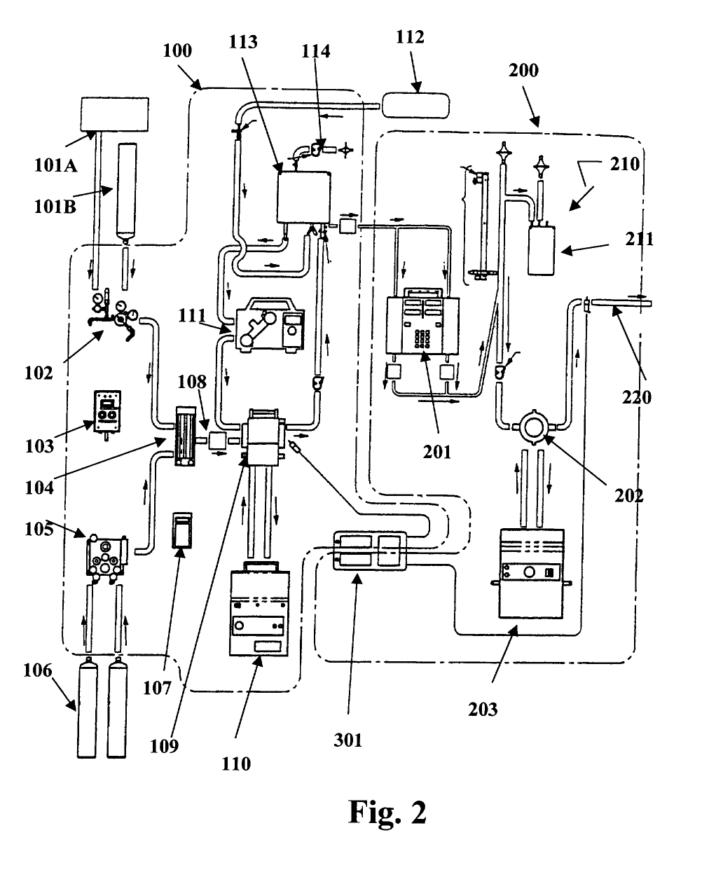

An exemplary perfusion device illustrated in FIG. 2 is made up of a conditioning circuit 100 and a delivery circuit 200. Oxygen is supplied by a wall oxygen supply 101A or an oxygen tank 101B, regulated by switching regulator 102. Carbon dioxide is provided by tanks 106, regulated by switching regulator 105. Gas supply can be monitored through indicia displayed on gas supply indicator 103. Gas flow can be monitored at rotameter 104. Gas is fed through filter 108 to hollow fiber oxygenator 108. An examples of a suitable oxygenator includes, for example, the Spinal OXY™ oxygenator from Baxter (Irvine, Calif.).

The fluorocarbon nutrient emulsion is introduced through port 112 to reservoir 113, which is vented by filtered vent 114. Circulation pump 111 delivers the fluorocarbon nutrient emulsion to the hollow fiber oxygenator 108 and an associated heating unit 110. The temperature of the fluorocarbon nutrient emulsion at the hollow fiber oxygenator 108 is monitored by temperature monitor 300. Fluorocarbon nutrient emulsion either cycles between reservoir 113 and the hollow fiber oxygenator 108/heating unit 110 or is delivered as needed to the delivery circuit 200 under the influence of delivery pump 201.

Delivery pump 201 delivers the fluorocarbon nutrient emulsion to heat exchanger 202 which is provided with heat exchange fluid by heat exchange conditioner 203. A pressure overlimit device 210, in this example a manometer-type device with fluid overflow at a pressure head that can be calibrated. Fluid overflow container 211 contains any overflow. Outlet 220 is to the inflow catheter. The temperature of fluorocarbon nutrient emulsion delivered to outlet 220 is also monitored by temperature monitor 300.

In one aspect of the invention, the conditioning circuit operates at the accepted physiological intracranial temperature of the animal being operated upon (+1° C.), while the delivery circuit lowers the temperature of the fluorocarbon nutrient emulsion or physiologically acceptable solution or suspension by at least 2° C., preferably by 2 to 27° C., more preferably by 5 to 15° C. Where a physiologically acceptable solution or suspension lacking an effective amount of oxygen-carrying compound is used, the conditioning can include temperature conditioning, providing the opportunity for the solution or suspension to outgas if needed, or, if bicarbonate-based buffer is used, conditioning with an appropriate concentration of carbon dioxide. This aspect of the invention can be used, for example, with simple saline solutions, but is preferably used with more sophisticated solutions or suspensions containing for example appropriate oncotic agents, or nutrient carbohydrate, or nutrient amino acids or precursors, or more sophisticated mixes of salts.

Further Information on Fluorocarbon Nutrient Emulsions

Further Information on fluorocarbon nutrient emulsions can be found, for example, in U.S. Pat. Nos. 4,378,797; 4,393,863; 4,446,154; 4,446,155; 4,657,532; 4,686,085; 4,758,431; 4,795,423; 4,830,849; 4,840,617; 4,963,130; 4,981,691; and 5,085,630, all to Jewell L. Osterholm.

### DEFINITIONS

The following terms shall have, for the purposes of this application, the respective meanings set forth below.

amino acid precursors. Amino acid precursors are compounds that are facilely converted by mammalian enzymes to a corresponding amino acid.

essentially lacking in an amino acid. A fluorocarbon nutrient emulsion or kit is essentially lacking in an amino acid if the amount is less than that which would reasonably be expected to provide an effective amount of nutrient. Such a lack exists when the concentration of the amino acid is 0.01 mg/L or 0.001 mg/L or less in the nutrient solution.

exposed cerebral-spinal tissue. Exposed cerebral-spinal tissue is any cerebral-spinal tissue which can be accessed by surgical equipment, including micro-scaled equipment such as endoscopes.

nutrient-providing effective amount. A nutrient-providing effective amount of a substance is a amount that can be expected, provided sufficient amounts of other nutrients, to increase metabolism or reproduction of mammalian cells compared with nutrient solutions lacking that substance.

oncotic agent. By oncotic agent is meant substances, generally macromolecules, that are of a size that is not readily able to leave the body cavity or other fluid containing body spaces (such as the cerebrospinal pathway, including the cerebral ventricles and subarachnoid spaces) into which they are inserted. Such oncotic agents are exemplified by blood plasma expanders which are known in general as macromolecules having a size sufficient to inhibit their escape from the blood plasma through the circulatory capillary bed into the interstitial spaces of the body. Serum albumin, preferably human serum albumin, is one well known blood plasma protein that can be used as an oncotic agent. Polysaccharide blood plasma expanders are often glucan polymers. For example, Hetastarch (a product of American Home Products) is an artificial colloid derived from a waxy starch composed almost entirely of amylopectin with hydroxyethyl ether groups introduced into the alpha (1-4) linked glucose units. The colloid properties of a 6% solution (wt/wt) of hetastarch approximates that of human serum albumin. Other polysaccharide derivatives may be suitable as oncotic agents in the blood substitute according to the invention. Among such other polysaccharide derivatives are hydroxymethyl alpha (1-4) or (1-6) polymers and cyclodextrins. In general, it is preferred that the polysaccharide is one that is non-antigenic. High molecular weight agents such as Dextran 70 having a molecular weight of about 70,000 Daltons are generally less preferred because they increase viscosity of the colloidal solution and impair the achievement of high flow rates. Preferably, the oncotic agent is in an amount effective to provide, in conjunction with other components of a fluorocarbon nutrient emulsion or a nutrient solution, an oncotic pressure of one to seven torr.

respiration. Respiration is the physical and chemical processes by which an organism supplies its cells and tissues with the oxygen needed for metabolism and, preferably, relieves them of the carbon dioxide formed in energy-producing reactions.

respiration-supporting amount. A respiration-supporting amount of oxygen is an amount that would, in model experiments, provide a statistically significant reduction in morbidity following a focal ischemic event.

All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

While this invention has been described with an emphasis upon preferred embodiments, it will be obvious to those of ordinary skill in the art that variations in the preferred devices and methods may be used and that it is intended that the invention may be practiced otherwise than as specifically described herein. Accordingly, this invention includes all modifications encompassed within the spirit and scope of the invention as defined by the claims that follow.

What is claimed:

1. A kit providing pre-measured amounts of components to form a fluorocarbon nutrient emulsion capable of carrying oxygen to living tissue, the kit comprising:
constituent solutions, emulsions or particle compositions, which are the constituent compositions, containing pre-measured amounts of components for making the fluorocarbon nutrient emulsion, the constituent compositions comprising:
poly-fluorinated, oxygen-carrying compound;
a physiologically acceptable emulsifying agent effective to emulsify the polymer;
a nutrient-providing effective amount of carbohydrate;
nutrient-providing effective amounts of amino acids or amino acid precursors;
an oncotic agent in amount effective to provide, in conjunction with the other components of the solution, a physiologically acceptable oncotic pressure; and
sufficient salts and buffering agents to provide a physiological osmotic pressure and physiologically appropriate concentrations of potassium and sodium ions;
wherein constituent compositions are selected to allow for sufficient stability of the components to allow for commercial marketing of the kit.

2. The kit of claim 1, wherein there are no more than eight constituent compositions.

3. The kit of claim 1, wherein there are no more than four constituent compositions.

4. The kit of claim 1, wherein at least three of constituent compositions are packaged together in separate chambers of a multi-chambered bag having pressure release seams separating the chambers, whereby pressure can be used to break the barriers between chambers to allow the contents to mix, wherein the contents mix to provide the appropriate concentrations.

5. The kit of claim 4, wherein the multi-chambered bag, or the multi-chambered bag together with a bag that envelops the multi-chambered bag has an carbon dioxide permeability of 10 cc/m²·day·atm or less.

6. The kit of claim 4, wherein the multi-chambered bag, or the multi-chambered bag together with a bag that envelops the multi-chambered bag has an carbon dioxide permeability of 1.0 cc/m²·day·atm or less.

7. The kit of claim 4, wherein the multi-chambered bag, or the multi-chambered bag together with a bag that envelops the multi-chambered bag has an carbon dioxide permeability of 0.5 cc/m²·day·atm or less.

8. The kit of claim 1, wherein the constituent compositions are adapted to provide a fluorocarbon nutrient emulsion with the following component amounts:

| | |
|---|---|
| Poly-Fluorinated, | 9.5-10-5 |
| Oxygen-Carrying | |
| Compound, % v/v | |
| Phospholipid, mg/mL | 11.5 |
| Albumin, g/dL, | 1.67 |
| α-Ketoglutaric Acid, μg/mL | 25 |
| Amino Acids, μg/mL | 17.5 |
| L-Isoleucine + L-Leucine | |
| L-Valine | 16.6 |
| L-Alanine | 28.6 |
| L-Serine | 24.6 |
| L-Histidine | 10.3 |
| L-Methionine | 2.1 |
| L-Phenylalanine + L-Lysine | 35.3 |
| L-Threonine + L-Arginine | 48.3 |
| L-Tyrosine | 7.9 |
| Na⁺, mM | 147 |
| K⁺, mM | 2.9 |
| Cl⁻, mM | 130 |
| Ca⁺², mM | 1.15 |
| Mg⁺², mM | 1.12 |
| Glucose (dextrose), mg/dL | 94 |

9. A kit providing pre-measured amounts of components to form a fluorocarbon nutrient emulsion capable of carrying oxygen to living tissue, the kit comprising: constituent solutions, emulsions or particle compositions, which are the constituent compositions, containing pre-measured amounts of components for making the fluorocarbon nutrient emulsion, the constituent compositions comprising:
a first constituent composition comprising an emulsion of poly-fluorinated, oxygen-carrying compound;
a second constituent composition comprising a solution of sodium and potassium salts;
a third constituent composition comprising a solution of a nutrient-providing effective amount of glucose;
a fourth constituent composition comprising a solution of an oncotic agent in amount effective to provide, in conjunction with the other components of the fluorocarbon nutrient emulsion, a physiologically acceptable oncotic pressure;
a fifth constituent composition comprising solution of nutrient-providing effective amounts of amino acids; and
a sixth constituent composition comprising a nutrient-providing effective amount of α-ketoglutaric acid.

10. The kit of claim 9, wherein the second constituent composition comprises one or both of calcium and magnesium salts.

11. The kit of claim 9, wherein the fifth constituent composition comprises nutrient-providing effective amounts of arginine, histidine, leucine, lysine, methionine, phenylalanine, threonine and valine, and all of the components are essentially lacking in glutamic acid, glutamine and glycine.

12. A kit providing pre-measured amounts of components to form a fluorocarbon nutrient emulsion capable of carrying oxygen to living tissue, the kit comprising: constituent solutions, emulsions or particle compositions, which are the constituent compositions, containing pre-measured amounts of components for making the fluorocarbon nutrient emulsion, the constituent compositions comprising:
a first constituent composition comprising an emulsion of poly-fluorinated, oxygen-carrying compound;
a second constituent composition comprising a solution of sodium, potassium, magnesium and calcium salts;
a third constituent composition comprising a solution of oncotic agent in an amount effective to provide, in conjunction with the other components of the fluorocarbon nutrient emulsion, a physiologically acceptable oncotic pressure; and
a fourth constituent composition comprising solution of a nutrient-providing effective amounts of amino acids,
wherein either the second constituent composition comprises a nutrient-providing effective amount of glucose or the kit comprises a fifth constituent composition comprising a nutrient-providing effective amount of glucose.

13. The kit of claim 12, wherein the first constituent composition comprises a nutrient-providing effective amount of α-ketoglutaric acid.

14. The kit of claim 12, wherein the fourth constituent composition comprises a nutrient-providing effective amount of α-ketoglutaric acid.

15. The kit of claim 12, wherein the second constituent composition comprises a nutrient-providing effective amount of glucose.

16. The kit of claim 15, wherein one or more of the second and fourth constituent compositions is in dried form adapted to be diluted in a pre-determined amount of water prior to use.

17. The kit of claim 12, wherein the kit comprises the fifth constituent composition.

18. The kit of claim 23, wherein one or more of the second, fourth and fifth constituent compositions is in dried form adapted to be diluted in a pre-determined amount of water prior to use.

19. The kit of claim 12, wherein at least the first, second and fourth constituent compositions are packaged together in separate chambers of a multi-chambered bag having pressure release seams separating the chambers, whereby pressure can be used to break the barriers between chambers to allow the contents to mix wherein the contents mix to provide the appropriate concentrations.

20. The kit of claim 19, wherein the multichambered bag has an injection port through which the third constituent composition can be injected to complete the fluorocarbon nutrient emulsion.

21. The kit of claim 12, wherein the first, second, third and fourth constituent compositions are packaged together in separate chambers of a multi-chambered bag having pressure release seams separating the chambers, whereby pressure can be used to break the barriers between chambers to allow the contents to mix, wherein the contents mix to provide the appropriate concentrations.

22. A fluorocarbon nutrient emulsion with the following component amounts:

| | |
|---|---|
| Poly-Fluorinated, | 9.5-10-5 |
| Oxygen-Carrying | |
| Compound, % v/v | |
| Albumin, g/dL, | 1.67 |
| α-Ketoglutaric Acid, μg/mL | 25 |
| Amino Acids, μg/mL | 17.5 |
| L-Isoleucine + L-Leucine | |
| L-Valine | 16.6 |
| L-Alanine | 28.6 |
| L-Serine | 24.6 |
| L-Histidine | 10.3 |
| L-Methionine | 2.1 |
| L-Phenylalanine + L-Lysine | 35.3 |
| L-Threonine + L-Arginine | 48.3 |
| L-Tyrosine | 7.9 |
| Na⁺, mM | 147 |
| K+, mM | 2.9 |
| Cl⁻, mM | 130 |
| Ca⁺², mM | 1.15 |
| Mg⁺², mM | 1.12 |
| Glucose (dextrose), mg/dL | 94 |

23. A vehicle kit providing pre-measured amounts of components to form a vehicle corresponding to a fluorocarbon nutrient emulsion formed from a corresponding fluorocarbon nutrient emulsion kit,
the corresponding fluorocarbon nutrient emulsion kit comprising constituent solutions, emulsions or particle compositions, which are the first constituent compositions, containing pre-measured amounts of components for making the fluorocarbon nutrient emulsion, the first constituent compositions made up of:
(a) poly-fluorinated, oxygen-carrying compound;
(b) a phospholipid emulsifying agent effective to emulsify the poly-fluorinated, oxygen-carrying compound, wherein the poly-fluorinated, oxygen-carrying compound and the phospholipid emulsifying agent are supplied in one first constituent composition wherein the poly-fluorinated, oxygen-carrying compound is emulsified by the phospholipid emulsifying agent, this emulsified poly-fluorinated, oxygen-carrying compound composition providing a portion of sodium or potassium ions of the fluorocarbon nutrient emulsion;
(c) a nutrient-providing effective amount of carbohydrate;
(d) nutrient-providing effective amounts of amino acids or amino acid precursors;
(e) an oncotic agent in amount effective to provide, in conjunction with the other components of the fluorocarbon nutrient emulsion, a physiologically acceptable oncotic pressure; and
(f) sufficient salts and buffering agents to provide a physiological osmotic pressure and physiologically appropriate concentrations of potassium and sodium ions;
the vehicle kit comprising the following separate vehicle kit compositions:
all the first constituent compositions but the emulsified poly-fluorinated, oxygen-carrying compound composition; and
supplement constituent compositions comprising one or more components effective to supply the sodium or potassium ions that would be provided by the emulsified poly-fluorinated, oxygen-carrying compound composition.

24. The vehicle kit of claim 23, wherein the vehicle kit compositions of the corresponding fluorocarbon nutrient emulsion kit comprise:
(1) a first constituent composition comprising an emulsion of poly-fluorinated, oxygen-carrying compound;
(2) a first constituent composition comprising a solution of sodium, potassium, magnesium and calcium salts;
(3) a first constituent composition comprising a solution of a nutrient-providing effective amount of glucose;
(4) a first constituent composition comprising a solution of an oncotic agent in amount effective to provide, in conjunction with the other components of the fluorocarbon nutrient emulsion, a physiologically acceptable oncotic pressure;
(5) a first constituent composition comprising a solution of nutrient-providing effective amounts of amino acids; and
(6) a first constituent composition comprising a nutrient-providing effective amount of α-ketoglutaric acid,
whereby the vehicle kit compositions comprise first constituent compositions (2) through (6) and at least one supplement constituent composition.

25. The vehicle kit of claim 23, wherein the vehicle kit compositions of the corresponding fluorocarbon nutrient emulsion kit comprise:
(1) a first constituent composition comprising an emulsion of poly-fluorinated, oxygen-carrying compound;
(2) a first constituent composition comprising a solution of sodium, potassium, magnesium and calcium salts;
(3) a first constituent composition comprising a solution of the oncotic agent in amount effective to provide, in conjunction with the other components of the fluorocarbon nutrient emulsion, a physiologically acceptable oncotic pressure; and
(4) a first constituent composition comprising a solution of a nutrient-providing effective amounts of amino acids,
whereby the vehicle kit compositions comprise first constituent compositions (2) through (4) and at least one supplement constituent composition.

26. The vehicle kit of claim 23, wherein the supplement constituent compositions are effective to supply the (α-ketoglutaric acid that would be provided by the emulsified poly-fluorinated, oxygen-carrying compound composition.

27. The vehicle kit of claim 23, wherein the supplement constituent compositions are effective to supply the phospholipid emulsifying agent that would be provided by the emulsified poly-fluorinated, oxygen-carrying compound composition.

28. A kit for use in delivering a fluorocarbon nutrient emulsion comprising (a) the vehicle kit of claim 23 and (b) the corresponding fluorocarbon nutrient emulsion kit.

29. The vehicle kit of claim 23, wherein vehicle kit compositions and supplement and one or more supplement compositions adapted to provide a vehicle solution with the following component amounts:

| | |
|---|---|
| Albumin, g/dL, | 1.67 |
| α-Ketoglutaric Acid, μg/mL | 25 |
| Amino Acids, μg/mL | 17.5 |
| L-Isoleucine + L-Leucine | |
| L-Valine | 16.6 |
| L-Alanine | 28.6 |
| L-Serine | 24.6 |
| L-Histidine | 10.3 |
| L-Methionine | 2.1 |
| L-Phenylalanine + L-Lysine | 35.3 |
| L-Threonine + L-Arginine | 48.3 |
| L-Tyrosine | 7.9 |
| Na⁺, mM | 147 |
| K⁺, mM | 2.9 |
| Cl⁻, mM | 130 |
| Ca⁺², mM | 1.15 |
| Mg⁺², mM | 1.12 |
| Glucose (dextrose), mg/dL | 94 |

30. The vehicle kit of claim 23, adapted to provide a vehicle solution with the following further component amount:

| | |
|---|---|
| Phospholipid, mg/mL | 11.5 |

31. The vehicle kit of claim 23, comprising:
a first vehicle kit composition comprising a solution of (i) sodium, potassium, magnesium and calcium salts, (ii) a nutrient-providing effective amount of α-ketoglutaric acid, and (iii) a nutrient-providing effective amounts of amino acids;
a second vehicle kit composition comprising a solution of the oncotic agent in amount effective to provide, in conjunction with the other components of the fluorocarbon nutrient emulsion, a physiologically acceptable oncotic pressure; and
a third vehicle kit composition comprising a solution of a nutrient-providing effective amount of glucose.

32. A vehicle solution with the following component amounts:

| | |
|---|---|
| Albumin, g/dL, | 1.67 |
| α-Ketoglutaric Acid, μg/mL | 25 |
| Amino Acids, μg/mL | 17.5 |
| L-Isoleucine + L-Leucine | |
| L-Valine | 16.6 |
| L-Alanine | 28.6 |
| L-Serine | 24.6 |
| L-Histidine | 10.3 |
| L-Methionine | 2.1 |
| L-Phenylalanine + L-Lysine | 35.3 |
| L-Threonine + L-Arginine | 48.3 |
| L-Tyrosine | 7.9 |
| Na⁺, mM | 147 |
| K⁺, mM | 2.9 |
| Cl⁻, mM | 130 |
| Ca⁺², mM | 1.15 |
| Mg⁺², mM | 1.12 |
| Glucose (dextrose), mg/dL | 94 |

33. A method of delivering a fluorocarbon nutrient emulsion to neural tissue of an animal having a cerebrospinal pathway, the method comprising:
(a) inserting a first catheter at a first point directed to deliver fluid to a cerebral ventricle;
(b) inserting a second catheter at a second point lower in the cerebrospinal pathway, which point is adapted to drain excess fluid due to fluid insertion through the first catheter;
(c) inserting through the first catheter a vehicle solution adapted to be physiologically compatible with the fluorocarbon nutrient emulsion, wherein the vehicle solution lacks sufficient oxygen carrying capacity to be capable of carrying a respiration-supporting amount of oxygen;
(d) confirming with the vehicle solution the existence of a perfusion pathway from the first catheter to the second catheter;
(e) if necessary, repositioning one or both of the catheters and repeating step (d) until a perfusion pathway is confirmed; and
(f) once a perfusion pathway is confirmed, inserting an oxygenated fluorocarbon nutrient emulsion through the first catheter.

34. A fluorocarbon nutrient emulsion capable of carrying oxygen to living tissue or a kit of pre-measured components for such a solution, the solution or kit comprising:
a poly-fluorinated, oxygen-carrying compound;
a physiologically acceptable emulsifying agent effective to emulsify the poly-fluorinated, oxygen-carrying compound; and
nutrient-providing effective amounts of amino acids or amino acid precursors, wherein the solution or kit is essentially lacking in glutamic acid, glutamine and glycine.

35. The fluorocarbon nutrient emulsion or kit of claim 34, further comprising a nutrient-providing effective amount of carbohydrate.

36. The fluorocarbon nutrient emulsion or kit of claim 34, wherein the nutrient-providing effective amounts of amino acids or amino acid precursors comprise a nutrient-providing effective amount of a precursor to glutamic acid or glutamine which is citric acid, cis-aconitic acid, isocitric acid, α-ketoglutaric acid, succinic acid, fumaric acid, malic acid or oxaloacetic acid or a pharmaceutically acceptable salt thereof.

37. A nutrient solution or a kit of pre-measured components for such a solution, the solution or kit comprising:
a nutrient-providing effective amount of carbohydrate;
an oncotic agent in amount effective to provide, in conjunction with the other components of the solution, a physiologically acceptable oncotic pressure; and
nutrient-providing effective amounts of amino acids or amino acid precursors including arginine, histidine, leucine, isoleucine, lysine, methionine, phenylalanine, threonine and valine, wherein the solution or kit is essentially lacking in glutamic acid, glutamnine and glycine.

38. The nutrient emulsion or kit of claim 37, wherein the nutrient-providing effective amounts of amino acids or amino acid precursors comprise a nutrient-providing effective amount of α-ketoglutaric acid or a pharmaceutically acceptable salt thereof.

39. A fluorocarbon nutrient emulsion capable of carrying a oxygen to living tissue or a kit of pre-measured components for such a solution, the solution or kit comprising:
a poly-fluorinated, oxygen-carrying compound;
a physiologically acceptable emulsifying agent effective to emulsify the poly-fluorinated, oxygen-carrying compound; and
nutrient-providing effective amounts of amino acids or amino acid precursors, including at least one citric acid, cis-aconitic acid, isocitric acid, succinic acid, fumaric acid, malic acid or oxaloacetic acid or a pharmaceutically acceptable salt thereof, wherein the solution or kit is essentially lacking in glutamic acid and glutamine.

40. A nutrient solution or a kit of pre-measured components for such a solution, the solution or kit comprising:
a nutrient-providing effective amount of carbohydrate;
an oncotic agent in amount effective to provide, in conjunction with the other components of the solution, a physiologically acceptable oncotic pressure; and
nutrient-providing effective amounts of amino acids or amino acid precursors including arginine, histidine, leucine, isoleucine, lysine, methionine, phenylalanine, threonine, valine, and at least one of citric acid, cis-aconitic acid, isocitric acid, succinic acid, fumaric acid, malic acid or oxaloacetic acid or a pharmaceutically acceptable salt thereof, wherein the solution or kit is essentially lacking in glutamic acid and glutamine.

41. A method of irrigating exposed cerebral-spinal tissue comprising irrigating with a solution comprising:
a nutrient-providing effective amount of carbohydrate;
an oncotic agent in amount effective to provide, in conjunction with the other components of the solution, a physiologically acceptable oncotic pressure; and
nutrient-providing effective amounts of amino acids or amino acid precursors including arginine, histidine, leucine, isoleucine, lysine, methionine, phenylalanine, threonine, valine, and at least one of citric acid, cis-aconitic acid, isocitric acid, α-ketoglutaric acid succinic acid, fumaric acid, malic acid or oxaloacetic acid or a pharmaceutically acceptable salt thereof, wherein the solution or kit is essentially lacking in glutamic acid and glutamine.

42. The method of claim 41, comprising conducting a surgery on cerebral-spinal tissue, the surgery comprising:
opening an animal to provide access to cerebral spinal tissue;
irrigating the accessed cerebral spinal tissue; and
conducting the surgery.

43. The method of claim 41, comprising conducting a surgery on cerebral-spinal tissue, the surgery comprising:
irrigating the exposed cerebral spinal tissue of an animal;
conducting the surgery; and
closing the animal to end the exposed state of the spinal tissue.

44. The method of claim 41, wherein the amino acids or precursors comprise tryptophan.

45. A method of delivering a physiologically acceptable solution or suspension to neural tissue of an animal having a cerebrospinal pathway, the method comprising:
conditioning the solution or suspension at temperature from within ±1° C. of the physiological intracranial temperature (T₀) of the animal;
reducing the temperature of the conditioned solution or suspension by at least 2° C.; and
perfusing the reduced temperature solution or suspension through at least a portion of the cerebral spinal pathway.

46. The method of claim 45, wherein the temperature of the conditioned solution or suspension is reduced by 2 to 27° C.

47. The method of claim 45, wherein the temperature of the conditioned solution or suspension is reduced by 5 to 15° C.

48. A method of claim 45 of delivering a fluorocarbon nutrient emulsion to neural tissue of an animal having a cerebrospinal pathway, the method comprising:
oxygenating an emulsion of a poly-fluorinated, oxygen-carrying compound at a temperature from within ±1° C. of the physiological intracranial temperature (T₀) of the animal;
reducing the temperature of the oxygenated emulsion by at least 2° C.; and
perfusing the reduced temperature emulsion through at least a portion of the cerebral spinal pathway.

49. The method of claim 48, wherein the temperature of the oxygenated emulsion is reduced by 2 to 27° C.

50. The method of claim 48, wherein the temperature of the oxygenated emulsion is reduced by 5 to 15° C.
